(19) United States
(12) Patent Application Publication
(10) Pub. No.: US 2026/0102441 A1
(43) Pub. Date: Apr. 16, 2026

(54) PROBIOTIC COMPOSITION FOR THE TREATMENT OF MALNUTRITION

(71) Applicant: Evonik Operations GmbH, Essen (DE)

(72) Inventors: Bodo SPECKMANN, Kahl (DE); Marco GOBBETTI, Bozen-Bolzano (IT); Heike TOM DIECK, Friedrichsdorf (DE)

(73) Assignee: Evonik Operations GmbH, Essen (DE)

(21) Appl. No.: 19/116,456

(22) PCT Filed: Sep. 15, 2023
(86) PCT No.: PCT/EP2023/075377

§ 371 Date: Mar. 28, 2025

(30) Foreign Application Priority Data
Sep. 30, 2022 (EP) 22198955.1

(51) Int. Cl. A61K 35/747; A23K 10/18; A61K 35/742; A61P 3/02

(52) U.S. Cl. CPC A61K 35/747; A23K 10/18; A61K 35/742; A61P 3/02

(57) ABSTRACT

The use of a probiotic composition includes the probiotic strains Lactobacillus plantarum (Lactiplantibacillus plantarum) DSM 33363, Lactobacillus plantarum (Lactiplantibacillus plantarum) DSM 33364, Lactobacillus paracasei (Lacticaseibacillus paracasei) DSM 33373, Lactobacillus reuteri (Limosilactobacillus reuteri) DSM 33374, Bacillus megaterium (Priestia megaterium) DSM 33300, Bacillus pumilus DSM 33297, Bacillus pumilus DSM 33355 (the “Pro-nutrient” consortium), for treating and preventing malnutrition of humans and animals by increasing the nutritional value of cereal-based foodstuffs, derived from wheat in particular, through increasing the bioavailability of essential micronutrients and amino acids contained in these foodstuffs.

This invention concerns probiotic compositions comprising the probiotic strains Lactobacillus plantarum (Lactiplantibacillus plantarum) DSM 33363, Lactobacillus plantarum (Lactiplantibacillus plantarum) DSM 33364, Lactobacillus paracasei (Lacticaseibacillus paracasei) DSM 33373, Lactobacillus reuteri (Limosilactobacillus reuteri) DSM 33374, Bacillus megaterium (Priestia megaterium) DSM 33300, Bacillus pumilus DSM 33297, Bacillus pumilus DSM 33355 (the “Pro-nutrient” consortium), for treating and preventing malnutrition of humans and animals by increasing the nutritional value of cereal-based foodstuffs, derived from wheat in particular, through increasing the bioavailability of essential micronutrients and amino acids contained in these foodstuffs.

Malnutrition, in the context of this invention, refers to deficiencies or imbalances in a person's intake of energy and/or nutrients, but not to overnutrition. Approximately 700 M people worldwide are undernourished, and considering the dimension of this problem, the World Health Organization has set ambitious Global nutrition targets for the year 2025 to e.g., target stunting, anemia, and low birth weight. To this end, the accessibility and quality of food needs to be improved, accompanied by technical solutions, as disclosed in this invention, that can maximize the nutritional value of foods.

While malnutrition is primarily a phenomenon of low- and middle-income nations, it is also prevalent in select groups of developed nations, e.g., the elderly, subjects affected by functional gastrointestinal disorders, vegetarians, vegans, and subjects practicing exclusion or unbalanced diets. Vegetarian, vegan, and wheat-based diets in particular go along with limited intake and bioavailability of the essential amino acid L-lysine and the essential micronutrients iron (Fe), zinc (Zn), and magnesium (Mg). Wheat-based diets also limit Fe, Zn, and Mg bioavailability by the presence of phytic acid, an anti-nutritional factor that chelates divalent cations and thereby prevents their absorption from the gut lumen. Large scale food fortification of e.g., table salt, soils and common crops has been applied, but despite such initiatives, micronutrient deficiencies continue to be highly prevalent and cause major global health issues [1].

Intake recommendations to meet the lysine requirement range from 64 to 30 mg/kg body weight per day [2]. Intake recommendations for Fe, Zn, and Mg range from 10 to 30 mg/day (Fe), 7 to 16 mg/day (Zn), and 300 to 350 mg/day (https://www.dge.de/wissenschaft/referenzwerte/). Interestingly, the recommendation for Zn is dependent on the intake of phytate.

The most common strategy to counteract existing or presumed micronutrient deficiencies is using dietary supplement or functional foods containing these micronutrients [3,4]. Supplementation, though, does not necessarily result in a satisfiable improved nutritional status, as the bioavailability of selected nutrients is affected by numerous intrinsic and extrinsic factors, such as interference with anti-nutritional factors, gut microbial factors, an individual's health status, and food matrix effects, with iron deficiency being a typical example [5]. On the other hand, excessive use of dietary supplements can lead to an oversupply and risk of adverse health outcomes, as indicated for beta carotene, vitamin A, and vitamin E [6]. In conclusion, possible limitations and risks of food supplementation include failure to improve the nutritional status of a given nutrients as well as the risk of exceeding the target range with possibly adverse health outcomes.

Deficiencies of macronutrients, e.g., protein are often addressed using enriched protein compositions, protein hydrolysates, peptides, or amino acid compositions with and without additional nutrients. WO2012052463 for example discloses the use of cysteine and derivatives thereof for the treatment and prevention of malnutrition. WO2019230849 discloses compositions of lysine, methionine together with minerals and vitamins as well as grains for treating malnutrition. Also, herbal and plant compositions have been described. For example, CN104623224 and CN105663996 describe compositions of traditional Chinese medicinal plants for treating e.g., infantile malnutrition.

This invention applies a microbiota-targeted strategy as a technical solution for improving the nutritional value of various diets and at the same time overcoming the limitations of sole supplementation strategies. The gastrointestinal microbiota determines the fate of orally ingested matter (diet, pharmaceuticals etc.) via e.g., microbial metabolization, interaction with host physiological functions such as barrier function, nutrient and water absorption, gastrointestinal motility, and in that sense is a crucial modulator of health in humans and animals. Microbiota-targeted strategies include the application of prebiotics, probiotics, synbiotics, and sometimes even fecal transplantations with the intention to modify the composition and activity of the microbiota. Probiotics are live microorganisms, which confer a health benefit on the host when administered in adequate amounts [7]. The most investigated and commercially available probiotics are mainly microorganisms from species of genera Lactobacillus and Bifidobacterium. In addition, several others such as Propionibacterium, Streptococcus, Bacillus, Enterococcus, Escherichia coli, and yeasts are also used. Different bacterial strains of the same genus and species may exert different effects on the host. A possible link between ingestion of Lactobacillus sp., Bifidobacterium sp, and Streptococcus thermophilus probiotics and status of the micronutrients vitamin B12, calcium, folate, iron and zinc has been described by meta-analysis of clinical trials conducted by Barkhidarian et al. [8]. Generally, the gut microbiome has been described as a confounder of the outcomes of nutritional intervention studies [9]. Lactic acid bacteria (LAB), including several Lactobacillus sp. express phytases. Use of these LAB has been described in the production of (fermented) foods like bread, soy milk, fruit juices, beer, and fermented vegetables. A prerequisite for a phytase functionality is sufficient survival of the LAB probiotic under gastrointestinal conditions. Only few reports have assessed phytase activities of LAB under such conditions; a screen of LAB isolates including Lactobacillus sp. revealed phytase activities ranging from ˜0.5 to maximal 1.77 U/ml, for a Weissella kimchii strain [10].

Recently, the taxonomic classification of several species of the genera Lactobacillus and Bacillus has been updated [11-13]. Of relevance in the context of this invention are the following species:

| “Old” denomination | Updated denomination (since 2020) |
|---|---|
| Lactobacillus paracasei | Lacticaseibacillus paracasei |
| Lactobacillus plantarum | Lactiplantibacillus plantarum |
| Lactobacillus reuteri | Limosilactobacillus reuteri |
| Bacillus megaterium | Priestia megaterium |
| Bacillus pumilus | Bacillus pumilus |

For convenience, for the example part the old denomination will be used, whereas both denominations will be used in the general description and in the claims section.

Under WO/2021/129998 and [14] we disclosed previously a combination of Lactobacillus plantarum (Lactiplantibacillus plantarum) DSM 33363, Lactobacillus plantarum (Lactiplantibacillus plantarum) DSM 33364, Lactobacillus paracasei (Lacticaseibacillus paracasei) DSM 33373, Lactobacillus reuteri (Limosilactobacillus reuteri) DSM 33374, Bacillus megaterium (Priestia megaterium) DSM 33300, Bacillus pumilus DSM 33297, Bacillus pumilus DSM 33355 (=the Pro-nutrient consortium), among other combinations [15], and its capability to fully digest gluten.

These strains have been deposited with the Leibniz-Institut DSMZ Deutsche Sammlung von Mikroorganismen and Zellkulturen GmbH, Inhoffenstr. 7B, 38124 Braunschweig, Germany in 2019 under the provisions of the Budapest Treaty on the International Recognition of the Deposit of Microorganisms for the Purpose of Patent Procedure under the Accession Number as mentioned before in the name of Evonik Nutrition and Care GmbH (legal predecessor of Evonik Operations GmbH).

Unexpectedly, we found out that the Pro-nutrient consortium has functionalities that makes it an effective treatment against malnutrition by improving the nutritional value of various foods and diets. We discovered that this consortium enables the release of the essential nutrients L-lysine, Fe, Zn, Mg from various food matrices (whole bread, white bread, wheat flour) upon simulated gastrointestinal digestion. This release is significantly stronger than under control conditions and as compared to the impact of other added digestive aids such as proteases and other probiotic consortia. We found that strains of the Pro-nutrient consortium have higher phytase activity than other probiotic strains, display very good survivability in simulated stomach and small intestinal conditions, and very good storage stability. These combined and unique features form a novel technical solution towards improving the nutritional value of various diets and thus to treat and/or prevent medical conditions arising from a lack of L-lysine, Fe, Zn, Mg, including anemia, fatigue, dizziness, nausea, dermatitis, low birth weight, growth retardation, stunting, and to improve the growth performance of animals.

Preparations of the Pro-nutrient consortium can be applied, for example, as a dietary supplement, feed additive, for the preparation of functional foods and feeds, as well as in the manufacture of food- and feedstuffs.

One advantage of our composition is that it can be integrated into the regular diet of a person, as compared to nutritional products such as formulated diets, medical nutrition, or functional foods, who (partially) replace the regular diet. Replacement diets can be inconvenient, costly, lack tastefulness, and be disadvantageous in social get-togethers. These disadvantages limit compliance and therefore the effectiveness of such treatments.

Therefore, the present invention is directed to a probiotic composition, wherein the probiotic composition comprises one or more of the following strains: Lactobacillus plantarum (Lactiplantibacillus plantarum) DSM 33363, Lactobacillus plantarum (Lactiplantibacillus plantarum) DSM 33363, Lactobacillus plantarum (Lactiplantibacillus plantarum) DSM 33364, Lactobacillus paracasei (Lacticaseibacillus paracasei) DSM 33373, Lactobacillus reuteri (Limosilactobacillus reuteri) DSM 33374, Bacillus megaterium (Priestia megaterium) DSM 33300, Bacillus pumilus DSM 33297, and Bacillus pumilus DSM 33355 for use in the treatment and prevention of malnutrition. More specifically, the preparation is suitable to improve the nutritional status of a person, especially the status of total protein, L-lysine, L-asparagine, L-glycine, L-ornithine, Fe, Zn, Mg, as determined by suitable biomarkers. Furthermore, the preparation is suitable to treat and prevent a deficiency or suboptimal status of any of the aforementioned nutrients, as well as to treat and prevent any health condition or disease arising from a chronic deficiency or suboptimal status of any of the aforementioned nutrients.

In a preferred configuration of the present invention, the probiotic composition comprises all of the strains Lactobacillus paracasei (Lacticaseibacillus paracasei) DSM 33373, Bacillus megaterium (Priestia megaterium) DSM 33300, Bacillus pumilus DSM 33297, and Bacillus pumilus DSM 33355. In another preferred configuration, the probiotic composition further comprises Lactobacillus plantarum (Lactiplantibacillus plantarum) DSM 33363, Lactobacillus plantarum (Lactiplantibacillus plantarum) DSM 33364, Lactobacillus reuteri (Limosilactobacillus reuteri) DSM 33374.

In a specific configuration, malnutrition is a deficiency of macronutrients and micronutrients, preferably a deficiency of proteins and minerals. More specifically, the probiotic composition reduces the content of phytic acid to increase the bioavailability of micronutrients selected from iron, copper, zinc and magnesium.

In another specific configuration, the probiotic composition has a phytase activity of at least 5 phytase activity units, preferably at least 10 phytase activity units, more preferably at least 15 phytase activity units.

Specifically, the probiotic composition is used to increase the nutritional value and/or the bioavailability of macronutrients and micronutrients in food products and in diets, preferably of proteins, amino acids, selected from L-lysine, L-aspartic acid, L-glycine and L-ornithine, or minerals, selected form Mg, Zn, Fe.

The cells of the strains of the current invention may be present in the compositions of the current invention, as spores (which are dormant), as vegetative cells (which are growing), as transition state cells (which are transitioning from vegetative cells to spores, or reverse), as cellular extracts or as a combination of at least two of these types of cells. In a preferred embodiment, the probiotic strain is present in a dormant form or as vegetative cells. In alternative embodiment, cytoplasmic extracts or cell-free supernatants or heat-killed biomass of the probiotic strains are used.

In an alternative embodiment, the preparations further comprise one or more probiotic strains.

In a further preferred embodiment, the preparations further comprise one or more of the following: microbial proteases purified from Aspergillus niger, Aspergillus oryzae, Bacillus sp., Lactobacillus sp., Pediococcus sp., Weissella sp., Rothia mucilaginosa, Rothia aeria, subtilisins, nattokinase.

In an alternative embodiment, the preparation further comprises enzymes that facilitate the digestion of carbohydrates, proteins, peptides, lipids.

In a preferred embodiment, the preparation for use further comprises a substance, which acts as permeabilizer of the microbial cell membrane of members of Bacillus sp., Lactobacillus sp., Pediococcus sp., Weissella sp., preferably alginate.

In an alternative embodiment, one or more of the probiotic strains selected from Bacillus sp. and Lactobacillus sp. are immobilized individually or as consortia. Immobilization can be realized on solid surfaces such as cellulose and chitosan, as entrapment within a porous matrix such as polysaccharide gels like alginates, k-carrageenan, agar, chitosan and polygalacturonic acid or other polymeric matrixes like gelatin, collagen, and polyvinyl alcohol or by flocculation and microencapsulation or electrospraying technologies.

One subject of the present invention is the use of a preparation according to the present invention as a food supplement or its use in foodstuffs. Preferred foodstuffs according to the invention are cereals, bread, chocolate products, gummies, mueslis, muesli bars, health bars, biscuits, spreads, and dairy products.

A further subject of the current invention is also the use of a preparation of the current invention as a synbiotic ingredient in food products.

One subject of the present invention is the use of a preparation according to the present invention as a food or feed supplement or functional food or food product or pharmaceutical product. Preferred foodstuffs according to the invention are cereals, bread, chocolate products, gummies, mueslis, muesli bars, health bars, biscuits, spreads, and dairy products.

Therefore, in a preferred embodiment, the preparation is formulated for oral use, preferably as pills, capsules, tablets, granular powders, opercula, soluble granules, bags, pills or drinkable vials, or is formulated as syrup or beverage, or is added to food, preferably cereals, gummies, bread, muesli, muesli bars, health bars, biscuits, chocolates, joghurts or spreads.

A further subject of the current invention is also the use of a preparation of the current invention as a synbiotic ingredient in food products.

A further subject of the present invention is a foodstuff composition containing a preparation according to the present invention and at least one further food ingredient, preferably selected from proteins, carbohydrates, fats, further probiotics, prebiotics, enzymes, vitamins, immune modulators, milk replacers, minerals, amino acids, coccidiostats, acid-based products, medicines, and combinations thereof. The foodstuff composition according to the present invention does also include dietary supplements, e.g. in the form of a pill, capsule, tablet, powder, sachet, opercula, soluble granules, bags, or drinkable vials, syrup, beverage, or other liquids.

A further subject of the current invention is a pharmaceutical composition containing a preparation according to the present invention and a pharmaceutically acceptable carrier.

Another subject of the current invention is the use as a feed additive to increase the feed conversion rate and reduce the luminal content of phytic acid to increase the bioavailability of one or more of the micronutrients iron, copper, zinc and magnesium and one or more of the amino acids L-lysine, L-aspartic acid, L-glycine and L-ornithine.

### WORKING EXAMPLES

### Example 1. Probiotic Composition Significantly Increases L-Lysine Release from Foodstuffs

Digests from three foodstuffs (whole bread, white bread, wheat flour protein) containing each 10 g of gluten proteins under simulated gastrointestinal conditions with and without addition of microbial consortia or proteases were prepared as described [14]. Protease 1 is a proline-specific oligopeptidase, protease 2 a caseine protease.

### L-Lysine Quantification:

Digests were assayed for the content of individual free amino acids (FAA) contained in the pH 4.6-soluble nitrogen fraction by a Biochrom 30 series amino acid analyzer (Biochrom Ltd., Cambridge Science Park, England) with a sodium cation-exchange column (20 by 0.46 cm [inner diameter]). A mixture of amino acids at known concentrations (Sigma Chemical Co., Milan, Italy) was added with tryptophan, ornithine, asparagine, and GABA and used as standard. Proteins and peptides in the samples were precipitated by addition of 5% (vol/vol) cold solid sulfosalicylic acid, holding the samples at 4° C. for 1 h, and centrifuging them at 15,000×g for 15 min. The supernatant was filtered through a 0.22-μm-pore-size filter and diluted, when necessary, with sodium citrate (0.2 M, pH 2.2) loading buffer. Amino acids were post-column derivatized with ninhydrin reagent and detected by absorbance at 440 (proline and hydroxyproline) or 570 (all the other amino acids) nm.

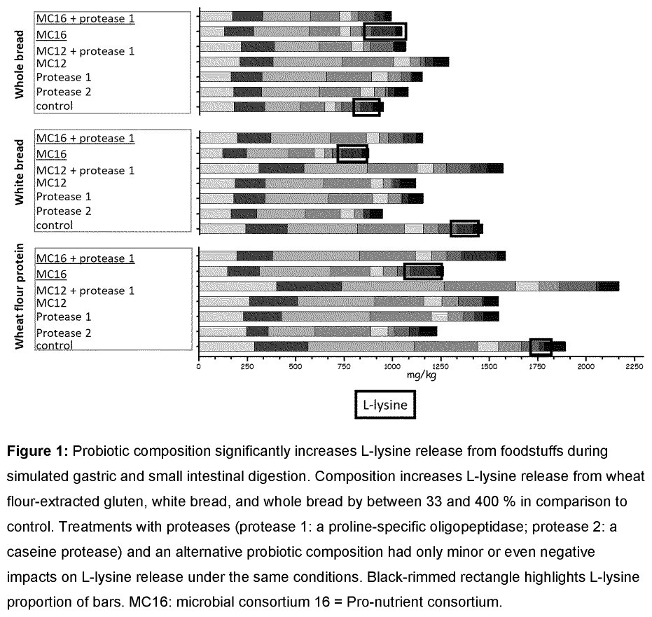

FIG. 1 shows that the probiotic composition significantly increases L-lysine release from foodstuffs during simulated gastric and small intestinal digestion. Composition increases L-lysine release from wheat flour-extracted gluten, white bread, and whole bread by between 33 and 400% in comparison to control. Treatments with proteases (protease 1: a proline-specific oligopeptidase; protease 2: a caseine protease) and an alternative probiotic composition had only minor or even negative impacts on L-lysine release under the same conditions. Black-rimmed rectangle highlights L-lysine proportion of bars. MC16: microbial consortium 16=Pro-nutrient consortium.

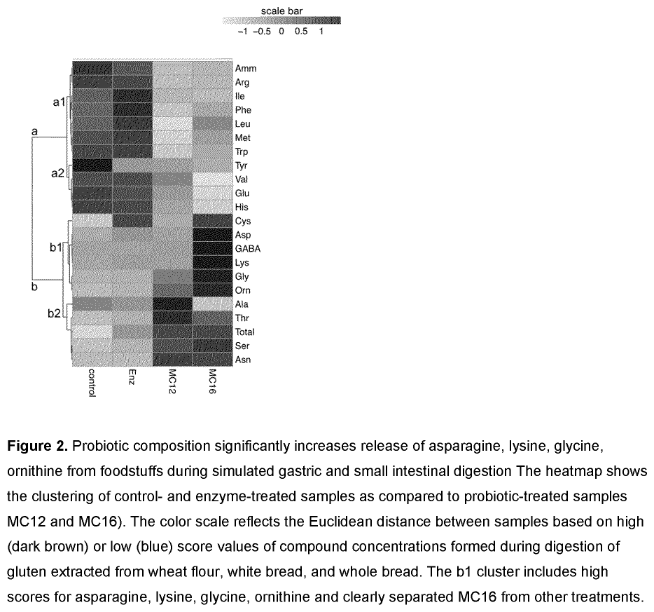

FIG. 2 shows that the probiotic composition significantly increases release of asparagine, lysine, glycine, ornithine from foodstuffs during simulated gastric and small intestinal digestion The heatmap shows the clustering of control- and enzyme-treated samples as compared to probiotic-treated samples MC12 and MC16). The color scale reflects the Euclidean distance between samples based on high (dark brown) or low (blue) score values of compound concentrations formed during digestion of gluten extracted from wheat flour, white bread, and whole bread. The b1 cluster includes high scores for asparagine, lysine, glycine, ornithine and clearly separated MC16 from other treatments.

FIGS. 1 and 2 show that the Pro-nutrient consortium (MC16) releases high amounts of the amino acids L-asparagine, L-lysine, L-glycine, L-ornithine from different foodstuffs during simulated gastric and small intestinal digestion. Importantly, this release is much more eminent as compared to control treatment and compared to another microbial consortium as well as two types of proteases.

### Example 2. Probiotic Strains Having Phytase Activity

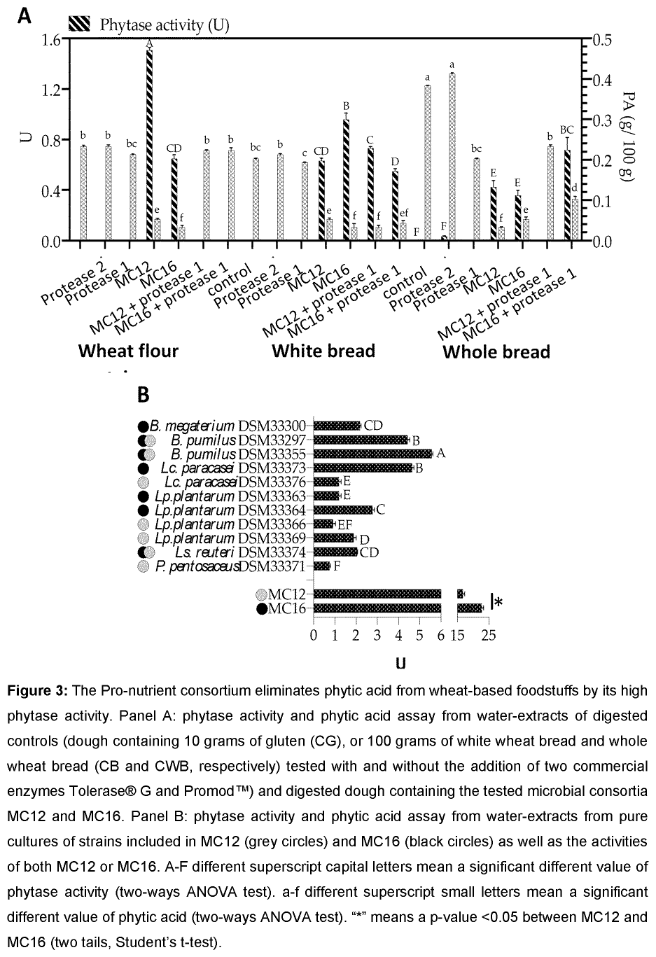

FIG. 3 shows that the Pro-nutrient consortium eliminates phytic acid from wheat-based foodstuffs by its high phytase activity. Panel A: phytase activity and phytic acid assay from water-extracts of digested controls (dough containing 10 grams of gluten (CG), or 100 grams of white wheat bread and whole wheat bread (CB and CWB, respectively) tested with and without the addition of two commercial enzymes Tolerase® G and Promod™) and digested dough containing the tested microbial consortia MC12 and MC16. Panel B: phytase activity and phytic acid assay from water-extracts from pure cultures of strains included in MC12 (grey circles) and MC16 (black circles) as well as the activities of both MC12 or MC16. A-F different superscript capital letters mean a significant different value of phytase activity (two-way ANOVA test). a-f different superscript small letters mean a significant different value of phytic acid (two-ways ANOVA test). “*” means a p-value <0.05 between MC12 and MC16 (two tails, Student's t-test).

FIG. 3 B displays phytase activities of individual strains of both consortia. Importantly, when the strains were combined to the two consortia, phytase activities were much higher, also in comparison to other wildtype probiotics referred to in the literature [16], with the Pro-nutrient consortium (MC16) having the highest activity of ˜24 U/ml.

Both consortia were applied in food digestion experiments under simulated gastrointestinal conditions to assess their capacity to reduce phytic acid (PA) content of wheat-based foodstuffs. As shown in FIG. 3, the Pro-nutrient consortium reduced PA of all tested foodstuffs by more than 84%; PA in wheat-derived gluten, white wheat bread, and whole wheat bread by 84%, 86, 1%, and 87, 1%, respectively. These reductions were much stronger than what has been reported elsewhere for other wildtype probiotic strains of e.g. the genus Lactobacillus [16, 17].

### LITERATURE

1. Keats, E. C.; Neufeld, L. M.; Garrett, G. S.; Mbuya, M. N. N.; Bhutta, Z. A. Improved micronutrient status and health outcomes in low- and middle-income countries following large-scale fortification: evidence from a systematic review and meta-analysis. Am J Clin Nutr 2019, 109, 1696-1708, doi:10.1093/ajcn/nqz023.
2. Tome, D.; Bos, C. Lysine requirement through the human life cycle. J Nutr 2007, 137, 1642S-1645S, doi:10.1093/jn/137.6.1642S.
3. Qato, D. M.; Alexander, G. C.; Conti, R. M.; Johnson, M.; Schumm, P.; Lindau, S. T. Use of prescription and over-the-counter medications and dietary supplements among older adults in the United States. JAMA 2008, 300, 2867-2878, doi:10.1001/jama.2008.892.
4. Kantor, E. D.; Rehm, C. D.; Du, M.; White, E.; Giovannucci, E. L. Trends in Dietary Supplement Use Among US Adults From 1999-2012. JAMA 2016, 316, 1464-1474, doi:10.1001/jama.2016.14403.
5. Kumar, S. B.; Arnipalli, S. R.; Mehta, P.; Carrau, S.; Ziouzenkova, O. Iron Deficiency Anemia: Efficacy and Limitations of Nutritional and Comprehensive Mitigation Strategies. Nutrients 2022, 14, doi:10.3390/nu14142976.
6. Bjelakovic, G.; Nikolova, D.; Gluud, L. L.; Simonetti, R. G.; Gluud, C. Mortality in randomized trials of antioxidant supplements for primary and secondary prevention: systematic review and meta-analysis. JAMA 2007, 297, 842-857, doi:10.1001/jama.297.8.842.
7. Hill, C.; Guarner, F.; Reid, G.; Gibson, G. R.; Merenstein, D. J.; Pot, B.; Morelli, L.; Canani, R. B.; Flint, H. J.; Salminen, S., et al. Expert consensus document. The International Scientific Association for Probiotics and Prebiotics consensus statement on the scope and appropriate use of the term probiotic. Nat Rev Gastroenterol Hepatol 2014, 11, 506-514, doi:10.1038/nrgastro.2014.66.
8. Barkhidarian, B.; Roldos, L.; Iskandar, M. M.; Saedisomeolia, A.; Kubow, S. Probiotic Supplementation and Micronutrient Status in Healthy Subjects: A Systematic Review of Clinical Trials. Nutrients 2021, 13, doi:10.3390/nu13093001.
9. Vandeputte, D. Personalized Nutrition Through The Gut Microbiota: Current Insights And Future Perspectives. Nutr Rev 2020, 78, 66-74, doi:10.1093/nutrit/nuaa098.
10. Andrabi, S. T.; Bhat, B.; Gupta, M.; Bajaj, B. K. Phytase-Producing Potential and Other Functional Attributes of Lactic Acid Bacteria Isolates for Prospective Probiotic Applications. Probiotics Antimicrob Proteins 2016, 8, 121-129, doi:10.1007/s12602-016-9220-3.
11. Zheng, J.; Wittouck, S.; Salvetti, E.; Franz, C.; Harris, H. M. B.; Mattarelli, P.; O'Toole, P. W.; Pot, B.; Vandamme, P.; Walter, J., et al. A taxonomic note on the genus Lactobacillus: Description of 23 novel genera, emended description of the genus Lactobacillus Beijerinck 1901, and union of Lactobacilloae and Leuconostocaceae. Int J Syst Evol Microbiol 2020, 70, 2782-2858, doi:10.1099/ijsem.0.004107.
12. Patel, S.; Gupta, R. S. A phylogenomic and comparative genomic framework for resolving the polyphyly of the genus Bacillus: Proposal for six new genera of Bacillus species, Peribacillus gen. nov., Cytobacillus gen. nov., Mesobacillus gen. nov., Neobacillus gen. nov., Metabacillus gen. nov. and Alkalihalobacillus gen. nov. Int J Syst Evol Microbiol 2020, 70, 406-438, doi:10.1099/ijsem.0.003775.
13. Gupta, R. S.; Patel, S.; Saini, N.; Chen, S. Robust demarcation of 17 distinct Bacillus species clades, proposed as novel Bacillaceae genera, by phylogenomics and comparative genomic analyses: description of Robertmurraya kyonggiensis sp. nov. and proposal for an emended genus Bacillus limiting it only to the members of the Subtilis and Cereus clades of species. Int J Syst Evol Microbiol 2020, 70, 5753-5798, doi:10.1099/ijsem.0.004475.
14. De Angelis, M.; Siragusa, S.; Vacca, M.; Di Cagno, R.; Cristofori, F.; Schwarm, M.; Pelzer, S.; Flugel, M.; Speckmann, B.; Francavilla, R., et al. Selection of Gut-Resistant Bacteria and Construction of Microbial Consortia for Improving Gluten Digestion under Simulated Gastrointestinal Conditions. Nutrients 2021, 13, doi:10.3390/nu13030992.
15. Lenhart, A.; Dong, T.; Joshi, S.; Jaffe, N.; Choo, C.; Liu, C.; Jacobs, J. P.; Lagishetty, V.; Shih, W.; Labus, J. S., et al. Effect of Exclusion Diets on Symptom Severity and the Gut Microbiota in Patients With Irritable Bowel Syndrome. Clin Gastroenterol Hepatol 2022, 20, e465-e483, doi:10.1016/j.cgh.2021.05.027.
16. Saraniya, A.; Jeevaratnam, K. In vitro probiotic evaluation of phytase producing Lactobacillus species isolated from Uttapam batter and their application in soy milk fermentation. J Food Sci Technol 2015, 52, 5631-5640, doi:10.1007/s13197-014-1686-y.
17. Amritha, G. K.; Venkateswaran, G. Use of Lactobacilli in Cereal-Legume Fermentation and as Potential Probiotics towards Phytate Hydrolysis. Probiotics Antimicrob Proteins 2018, 10, 647-653, doi:10.1007/s12602-017-9328-0.

1. A probiotic composition comprising one or more of the following strains: Lactobacillus plantarum (Lactiplantibacillus plantarum) DSM 33363, Lactobacillus plantarum (Lactiplantibacillus plantarum) DSM 33364, Lactobacillus paracasei (Lacticaseibacillus paracasei) DSM 33373, Lactobacillus reuteri (Limosilactobacillus reuteri) DSM 33374, Bacillus megaterium (Priestia megaterium) DSM 33300, Bacillus pumilus DSM 33297, and Bacillus pumilus DSM 33355 for use in the treatment or prevention of a deficiency of total protein, L-lysine, L-asparagine, L-glycine, L-ornithine, or minerals selected from Fe, Zn or Mg.

2. The composition according to claim 1, wherein the probiotic composition comprises all of the strains Lactobacillus paracasei (Lacticaseibacillus paracasei) DSM 33373, Bacillus megaterium (Priestia megaterium) DSM 33300, Bacillus pumilus DSM 33297, and Bacillus pumilus DSM 33355.

3. The composition according to claim 1, wherein the probiotic composition further comprises Lactobacillus plantarum (Lactiplantibacillus plantarum) DSM 33363, Lactobacillus plantarum (Lactiplantibacillus plantarum) DSM 33364, Lactobacillus reuteri (Limosilactobacillus reuteri) DSM 33374.

4. The composition according to claim 1, wherein the probiotic composition has a phytase activity of at least 5 phytase activity units, preferably at least 10 phytase activity units, more preferably at least 15 phytase activity units.

5. The composition according to claim 1, wherein the probiotic composition is a food or feed supplement or functional food or food product or pharmaceutical product.

6. The composition according to claim 5, wherein said probiotic composition is formulated for oral use, preferably as pills, capsules, tablets, granular powders, opercula, soluble granules, bags, pills or drinkable vials, or is formulated as syrup or beverage, or is added to food, preferably cereals, gummies, bread, muesli, muesli bars, health bars, biscuits, chocolates, joghurts or spreads.

7. The composition according to claim 5 as a feed additive to increase the feed conversion rate and reduce the luminal content of phytic acid to increase the bioavailability of one or more of the micronutrients iron, copper, zinc and magnesium and one or more of the amino acids L-lysine, L-aspartic acid, L-glycine and L-ornithine.
